(19) United States
(12) Patent Application Publication
(10) Pub. No.: US 2019/0117634 A1
(43) Pub. Date: Apr. 25, 2019

(54) Treatment of Vulvodynia

(71) Applicant: Gruenenthal GmbH, Aachen (DE)

(72) Inventors: Emilio QUETGLAS, Cabanillas del Campo (ES); Peter HEIN, Aachen (DE); Wolfgang SCHRÖDER, Aachen (DE); Andreas SCHOLZ, Giessen (DE)

(21) Appl. No.: 16/156,775

(22) Filed: Oct. 10, 2018

(30) Foreign Application Priority Data
Oct. 17, 2017 (EP) 17196903.3

(51) Int. Cl. A61K 31/438; A61K 9/00; A61P 15/02

(52) U.S. Cl. CPC A61K 31/438; A61P 15/02; A61K 9/0034

(57) ABSTRACT

The invention relates to a pharmaceutical composition comprising cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine. The pharmaceutical composition is suitable for local treatment of vulvar pain such as vulvodynia.

This application claims foreign priority benefit of European Application No. EP 17196903.3, filed Oct. 17, 2017, the disclosure of which patent application is incorporated herein by reference.

The invention relates to a pharmaceutical composition comprising cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine. The pharmaceutical composition is suitable for local treatment of vulvar pain such as vulvodynia.

Women can experience different forms of vulvar pain such as itching, burning, feeling raw or experience a splitting sensation. Vulvar pain can also include hypersensitivity on touch, such as during intercourse and on insertion of tampons. The degree of pain experienced can be so severe that sitting down or even wearing trousers can be painful. Vulvar pain can affect women at any age.

There are numerous causes for vulvar pain, which the International Society for the Study of Vulvovaginal Disease classifies in two diagnostic groups:

(1) Vulvar pain related to a specific disorder; and

### (2) Vulvodynia.

Vulvar pain related to a specific disorder includes specific disorders causing vulvar pain such as infectious conditions (e.g. vulvar candidiasis, herpes, etc.), inflammatory conditions (e.g. lichen sclerosus, lichen planus, atrophic vaginitis, immunobullous disorder), neoplastic conditions (e.g. Paget's disease, squamous cell carcinoma, vulvar intraepithelial neoplasia), neurological conditions (e.g. herpes neuralgia, spinal nerve compression), or hormonal disbalance.

Vulvodynia differs from vulvar pain related to a specific disorder in that the pain cannot be related to a specific cause, i.e. there is no specific aetiology. Vulvodynia has been defined by the International Society for the Study of Vulvovaginal Disease as “vulvar discomfort, most often described as a burning pain, occurring in the absence of relevant visible findings or a specific, clinically identifiable neurologic disorder”. Further definitions are provided by the International Pelvic Pain Society in J. B. Bornstein et al., “2015 Consensus terminology and classification of persistent vulvar pain”.

Vulvodynia can be classified by the anatomical site of the pain. When the pain affects the entire vulva this vulvodynia is referred to as generalized vulvodynia. When the pain affects parts of the vulva such as the vestibule, the clitoris and/or other portions of the vulva this vulvodynia is referred to as localized vulvodynia. Localized vulvodynia includes disorders such as vestibulodynia, clitorodynia, and hemivulvodynia. Some patients suffer from mixed vulvodynia, which includes presence of generalized and localized forms in the same patient. Both generalized vulvodynia and localized vulvodynia can be provoked, unprovoked, or mixed (i.e. provoked and unprovoked). Provoked vulvodynia includes sexual provocation of pain, non-sexual provocation of pain or both.

The cause for vulvodynia symptoms, which include sensations like burning, stabbing, shooting, aching or like an electric shock, are elusive. Various theories suggest a multifactorial origin of vulvodynia. It is often observed that vulvodynia appears after fungal and/or bacterial infections, urinary tract infections, postoperative infections or local injury. There are assumptions that pain, e.g. caused by an inflammatory condition, which lasts more than 3-6 months sensitizes the central and/or peripheral nervous system. This sensitization may be responsible for the perpetuation of symptoms once the original cause for the pain has resolved.

Vulvodynia is a chronic pain syndrome. Further, vulvodynia may be a neuropathic pain syndrome and has neuropathic pain characteristics.

Currently there are several treatment options for vulvar pain (vulvodynia) including physiotherapy, sex therapy, psychological therapy, systemic pharmacological therapy, intralesional therapy, topical therapy and even surgical treatment which usually are combined.

Commonly employed drugs in systemic therapy of vulvar pain (vulvodynia) are tricyclic antidepressants such as amitriptyline, imipramine, nortriptyline and desipramine. Some tricyclic antidepressants may help improve pain control, in particular neuropathic pain, such as amitriptyline. Also pain modifying drugs which are prescribed for pain relief from neuropathic pain and chronic pain such as gabapentin or pregabalin are employed in the treatment of vulvar pain. Other employed drugs are selective serotonine reuptake inhibitors and serotonin and noradrenalin reuptake inhibitors. However, the drugs commonly prescribed for the treatment of vulvar pain have considerable side effects such as sedation, dry mouth, constipation, dizziness, weight gain and cognitive impairment. Further, studies have shown that only 47% of women taking amitriptyline report a complete pain relief (D. Nunns, “Vulvodynia Management”, Obstetrics, Gynecology and Reproductive Medicine, 2015, 25:3, pages 68-74). Overall, none of the used pharmacological treatments has been tested as effective in this condition thus far in a confirmatory and adequately controlled clinical trial.

Intralesional therapy includes the local injection of drugs such as steroids, botulinum toxin, betamethasone and lidocaine, wherein the injection is usually in the vestibular epithelium of the vulva. One of the functions of epithelial tissue is to provide sensation. The long-term effects of such treatments are unclear. Further, injections in the region of the vulva go along with additional pain.

Topical therapy includes primarily application of local anesthetics such as lidocaine or pain killers such as gabapentin. In topical therapy the drugs are applied as a gel or an ointment comprising the drug in different concentrations. However, when local anesthetics are applied topically they can sting so that this treatment is uncomfortable for women. When topical treatments comprising local anesthetics are employed prior to sexual intercourse partners can become numb with sexual intercourse.

Further, the skin of the vulva is irritated more easily than the skin elsewhere as the stratum corneum of the vulvar skin functions less efficiently as a protective barrier, so that topical agents may cause unwanted side effects such as allergic reactions more easily. Also, often women suffering from vulvar pain may have employed various topical agents in the past, such as prescription based treatments against e.g. fungi, special soaps, baths and hygiene-sprays; so that the skin of the vulva may be already irritated before topical treatment of vulvodynia starts. Furthermore, application of a gel or ointment may be painful, especially for women whose symptoms include hypersensitivity on touch.

Surgical treatment is taken into consideration after the other treatment options are exhausted. The currently practiced technique is modified vestibulectomy. However, postoperative complications may occur such as decreased vaginal lubrication and even worsening of pain. Additionally, surgical treatment involves complications and risks connected to anesthesia, the surgery itself, e.g. damages of the urinal tract, and postoperative complications, e.g. hurting or ugly scars.

US 2003/0162769 discloses a medicament for the treatment of vulvodynia. The medicament is in the dosage form of a vaginal suppository, and the primary active ingredient is a calcium antagonist, diltiazem hydrochloride in the preferred embodiment.

US 2004/0198775 relates to methods of using Cav2.2 subunit calcium channel modulators to treat painful and non-painful lower urinary tract disorders and the related genitourinary tract disorders, vulvodynia and vulvar vestibulitis in normal and spinal cord injured patients.

US 2007/0049627 discloses methods of using prodrugs of GABA analogs and pharmaceutical compositions thereof to treat vulvodynia in a patient, and pharmaceutical compositions of prodrugs of GABA analogs useful in treating vulvodynia.

The treatment options for vulvar pain, especially vulvodynia according to the prior art are not satisfactory in every respect and there is a demand for new medicaments for treating vulvodynia.

It is an object of the invention to provide medicaments that are useful for ameliorating conditions and symptoms that are associated with vulvar pain, especially for treating vulvar pain such as vulvodynia, potentially accompanied by hyperalgesia, allodynia, discomfort, hypersensitivity, and the like, and that have advantages compared to the prior art.

These objects have been achieved by the subject-matter as described hereinbelow.

A first aspect of the invention relates to a pharmacologically active compound according to the invention for use in the treatment of vulvar pain, wherein the pharmacologically active compound according to the invention is cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or a physiologically acceptable salt thereof.

The pharmacologically active compound according to the invention cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′ [1H]-pyrido[3,4-b]indol]-4-amine is an analgesic known from WO 2012/013343.

The pharmacologically active compound according to the invention is cis-(E)-4-(3-fluorophenyl)-2′,3′,4′,9′-tetrahydro-N,N-dimethyl-2′-(1-oxo-3-phenyl-2-propenyl)-spiro[cycyclo-hexane-1,1′ [1H]-pyrido[3,4-b]indol]-4-amine having the following structure

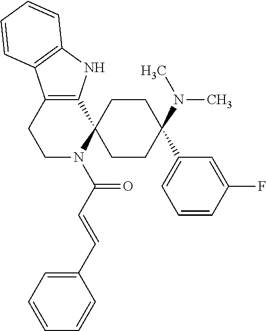 [SMILES: CN(C)[C@]1(c2cccc(F)c2)CC[C@]2(CC1)c1[nH]c3ccccc3c1CCN2C(=O)/C=C/c1ccccc1]

or a physiologically acceptable salt thereof.

Physiologically acceptable salts of the pharmacologically active compound according to the invention include but are not limited to the citrate salt and the hydrochloride salt. Preferably, the pharmacologically active compound according to the invention is present in the non-salt form, i.e. in form of its free base. Nonetheless, a skilled person recognizes that depending upon the pH value of a pharmaceutical composition containing the pharmacologically active compound according to the invention and its constituents, acid addition salts may form in situ. In the course of the preparation of such pharmaceutical compositions, the pharmacologically active compound according to the invention is preferably added in the non-salt form, i.e. in form of its free base.

The pharmacologically active compound according to the invention exhibits activity, for example, on the ORL1 receptor (also referred to as “NOR receptor” or “nociception-orphanin FQ peptide receptor”), which is relevant in connection with various diseases and which inter alia plays a role in analgesia.

There is indication that cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or a physiologically acceptable salt thereof has advantageous effects in the treatment of vulvar pain. Further, it has been surprisingly found that cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or a physiologically acceptable salt thereof permeate or penetrate into the epithelium of vulvar tissue. Without wishing to be bound to any scientific theory it is believed that penetration of the inventive pharmacologically active compound according to the invention into the epithelium of vulvar tissue leads to relief from vulvar pain, in particular relief from pain caused by vulvodynia.

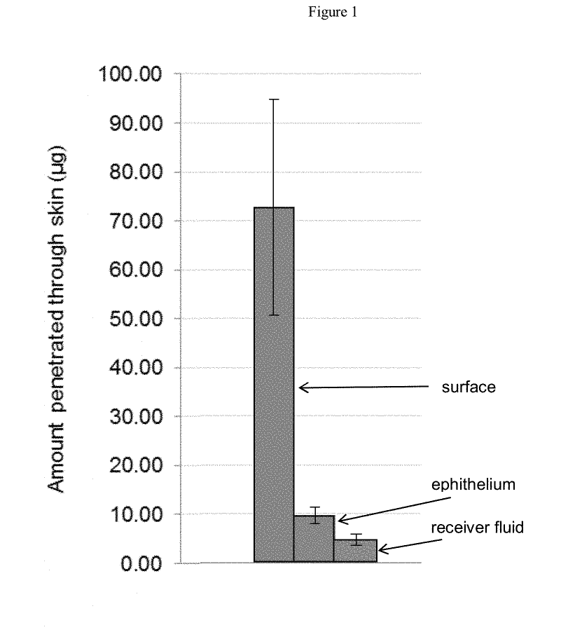

FIG. 1 illustrates the results ofpermeation/penetration in vitro experiments carried out with a formulation comprising the pharmacologically active compound according to the invention.

In a preferred embodiment, the pharmacologically active compound according to the invention is for use in the treatment of vulvar pain, wherein the vulvar pain is preferably related to or associated with
an infectious disorder, preferably selected from the group consisting of vulvar candidiasis and herpes;
an inflammatory disorder, preferably selected from the group consisting of lichen sclerosus, lichen planus, atrophic vaginitis (vaginal atrophy) and immunobullous disorder;
a neoplastic disorder, preferably selected from the group consisting of Paget's disease, vulvar intraepithelial neoplasia and squamous cell carcinoma;
a neurologic disorder, preferably selected from herpes neuralgia and spinal nerve compression;
dyspareunia; or
hormonal disbalance.

For the purpose of the specification, the term “vulvar pain” preferably is a condition selected from the group consisting of vaginal pain, hyperalgesia, allodynia, discomfort, hypersensitivity, and mixtures thereof. Further, for the purpose of the specification “vulvodynia” is potentially accompanied by hyperalgesia, allodynia, discomfort, hypersensitivity, and the like. Therefore, for the purpose of the specification, the treatment of vulvodynia may also involve the treatment of accompanying conditions such as hyperalgesia, allodynia, discomfort, hypersensitivity, and the like, respectively.

In a preferred embodiment, the vulvar pain is central vulvar pain. In another preferred embodiment, the vulvar pain is peripheral vulvar pain.

In a preferred embodiment, the vulvar pain is acute vulvar pain. In another preferred embodiment, the vulvar pain is chronic vulvar pain.

In a particularly preferred embodiment, the pharmacologically active compound according to the invention is for use in the treatment of vulvodynia. Preferably, the vulvodynia is generalized vulvodynia, localized vulvodynia, or mixtures thereof. Preferably, the vulvodynia is localized vulvodynia selected from the group consisting of vestibulodynia (vulvar vestibulitis), clitorodynia, hemivulvodynia, and pain in other locations, e.g. pain localized in the labia minora. Preferably, the pharmacologically active compound according to the invention is for use in the treatment of provoked vulvodynia, unprovoked vulvodynia, or mixtures thereof.

Preferably, the pharmacologically active compound according to the invention is applied topically.

Preferably, the pharmacologically active compound according to the invention is administered locally, more preferably the pharmacologically active compound according to the invention is administered on the vulva or on regions thereof.

In a preferred embodiment, especially when the pharmacologically active compound according to the invention is for use in the treatment of generalized vulvodynia, the pharmacologically active compound according to the invention is administered on the entire vulva. In a preferred embodiment, the pharmacologically active compound according to the invention is administered to the non-keratinized mucosa in the vulva.

In another preferred embodiment, especially when the pharmacologically active compound according to the invention is used in the treatment of localized vulvodynia, the pharmacologically active compound according to the invention is administered only on the affected parts of the vulva, i.e. the vestibule, the clitoris and/or other portions of the vulva.

Preferably, the pharmacologically active compound according to the invention is applied in form of a solution, dispersion, emulsion, suspension, or a mixture thereof.

Preferably, the pharmacologically active compound according to the invention is formulated in form of a liquid, a gel, an ointment, a creme, or a lotion.

Preferably, the pharmacologically active compound according to the invention is provided in form of a pharmaceutical composition from which the pharmacologically active compound according to the invention permeates into the epithelium of the vulvar tissue.

The invention also relates to a pharmaceutical composition comprising the pharmacologically active compound according to the invention and at least one suitable additive and/or auxiliary substance and/or optionally further pharmacologically active compounds.

In addition to the pharmacologically active compound according to the invention, the pharmaceutical composition according to the invention optionally comprises suitable additives and/or auxiliary substances, e.g. carriers, fillers, solvents, diluents, dyestuffs and/or binders, and can be administered as liquid medicament form, e.g. in the form of an injection solution, drops or a juice; or as semi-solid medicament form, e.g. in the form of granules, tablets, pellets, patches, capsules, plasters/spray plasters or aerosols. Further, the pharmaceutical composition according to the invention can be administered in the form of liquids, gels, ointments, cremes, or lotions.

Preferably, the pharmaceutical composition comprises the pharmacologically active compound according to the invention in form of a liquid, a gel, an ointment, a creme, or a lotion.

The amount of the pharmacologically active compound according to the invention to be administered to the patient varies according to the weight of the patient, the mode of administration, the indication and the severity of the disease. 0.00005 to 50 mg/kg, preferably 0.001 to 0.5 mg/kg of the pharmacologically active compound according to the invention are conventionally administered.

Another aspect of the invention relates to a pharmaceutical composition according to the invention as described above for use in the amelioration of conditions and symptoms that are associated with vulvar pain, especially for use in the treatment of vulvodynia. In this regard, the invention also pertains to the use of the pharmacologically active compound according to the invention for the manufacture of the pharmaceutical compositions, in particular liquids, gels, ointments, cremes, or lotions according to the invention as described above for use in the amelioration of conditions and symptoms that are associated with vulvar pain, especially for use in the treatment of vulvodynia.

Further, the invention also pertains to a method for ameliorating conditions and symptoms that are associated with vulvar pain, especially for treating vulvodynia, comprising administering to a subject in need thereof the pharmacologically active compound according to the invention and the pharmaceutical compositions according to the invention, respectively, as described above.

Preferably, the pharmaceutical composition according to the invention is administered topically. Preferably, the pharmaceutical composition according to the invention is administered locally, more preferably on the vulva or on regions thereof.

Preferably, the pharmacologically active compound according to the invention and the pharmaceutical composition according to the invention, respectively, is administered once, twice or three times a day, as required. A skilled person recognizes that the frequency of application depends on the administered dose of the pharmacologically active compound according to the invention, e.g. on the concentration of the pharmacologically active compound according to the invention in the pharmaceutical composition, and on the intensity of pain experienced by the patient. Preferably, the pharmacologically active compound according to the invention and the pharmaceutical composition according to the invention, respectively, is administered every 2 hours, or every 3 hours, more preferably every 4 hours, or every 5 hours, even more preferably every 6 hours, most preferably every 12 hours, in particular every 24 hours.

In another preferred embodiment, the pharmacologically active compound according to the invention and the pharmaceutical composition according to the invention, respectively, is administered less frequently than once daily, e.g. every second day or once a week.

The following examples further illustrate the invention but are not to be construed as limiting its scope:

Unless expressly stated otherwise, all percentages are wt.-%. Further, unless expressly stated otherwise, all weights and percentages of the API are expressed in terms of equivalents relative to the weight of the non-salt form of the API. Unless expressly stated otherwise, all properties are determined at 50% relative humidity and 23° C.

### EXAMPLE 1

A topical formulation was prepared comprising the ingredients summarized in the following table:

| excipient (wt.-%) | content |
|---|---|
| Ethanol | 15.00 |
| SR Polyethylene glycol 400 | 66.30 |
| Diisopropyl adipate | 15.00 |
| Butylated hydroxytoluene | 0.10 |
| Hydroxypropyl cellulose HF | 1.00 |
| Pharmacologically active compound according to the invention | 2.60 |
| Total | 100.00 |

### EXAMPLE 2

The permeation/penetration of the formulation according to Example 1 in porcine vaginal tissue was assessed by in vitro experiments.

The in vitro experiments involved the use of a Franz diffusion cell designed to mimic the physiological and anatomical conditions of porcine vaginal tissue in situ. Porcine vaginal tissue skin was positioned between the two halves of a Franz diffusion cell with the vaginal epithelium facing the donor compartment allowing for drug product application. The other side of the porcine vaginal tissue skin faced a receiver fluid comprising 2% w/v Tween 80 in 20% v/v PEG 400 in water. The Franz cells employed had an average surface area of approximately 0.6 cm² and a volume of approximately 2.0 mL.

Prior to dosing of the formulation on the tissue, the integrity of the tissue was assessed as follows:
(i) Porcine vaginal tissue was mounted between the donor and receiver compartments and the cells were sealed together using Parafilm®.
(ii) The donor and receiver chambers were filled with PBS solution and a small magnetic follower was placed in the receiver compartment.
(iii) Cells were equilibrated in a water bath, ensuring a membrane temperature of 32° C. for 30 min.
(iv) The electrodes of the ISO-TECH LCR821 Meter were placed in the receiver chamber through the sampling arm and the donor chamber and the LCR was set at 100 Hz and set to ‘R’ for resistance.
(v) The resistance of the tissue in each Franz cell was measured using a ISO-TECH LCR821 Meter (SOP 3174). Cells with a resistance below the acceptable limits were discarded and remounted.

Following the tissue integrity testing, the PBS solutions were removed from each compartment and the receiver compartments of cells passing the resistance criteria were filled with receiver fluid. Each cell was then equilibrated to ensure a surface temperature of 32° C. (external skin surface temperature) for at least 30 min prior to dosing. An additional Franz cell was also mounted but not dosed (to act as a blank) to assess interference with sample quantification.

A positive displacement pipette was used to apply 6-8 mg of the formulation of table 1 to the tissue surface. Prior to application the weight of each test formulation was verified by weighing the amount dispensed by the positive displacement pipette into an empty vial (n=6).

Receiver fluid (200 μL) was removed at the following time points t=0, 1, 2, 4, 6 and 24 h and transferred to a HPLC vial for analysis. Fresh pre-warmed (37° C.) receiver fluid was used to replace the receiver fluid removed at each time point. Following the 24 h time point, remaining receiver fluid was removed from the Franz cells and the drug was recovered.

The recovery of the residual formulation on vaginal tissue was performed as follows:
(i) A total of three cotton swabs were used to recover the drug from the surface of the porcine vaginal tissue.
(ii) After dismantling the donor chamber from the Franz cell, one dry cotton swab (Johnson's Cotton Wool Buds; Johnson & Johnson, UK) was used to remove all of the formulation from the surface of the porcine vaginal tissue and the swab placed into the 7 mL vial.
(iii) A second swab was then immersed into an extraction diluent (90:10 v/v ethanol:water) and used to swab the surface of the porcine vaginal tissue; this swab was then placed into the vial containing the first swab.
(iv) The final swab was used dry to swab the surface of the porcine vaginal tissue and then placed into the glass vial containing the two other swabs (Step (iii)).
(v) An initial tape strip from the surface of the porcine vaginal tissue was placed in a separate vial to the cotton swabs (Steps (iv)) and 2 mL of extraction diluent was added.
(vi) Each vial was then shaken on an orbital shaker at ambient temperature for 16-20 h in the extraction diluent to facilitate the extraction.
(vii) Following the extraction procedure, (Step (vi)), the extraction diluent was removed from the vials and centrifuged at ca. 16,060 g-force for 10 min to remove all un-dissolved materials and particles.
(viii) The supernatant from each sample was transferred to a HPLC vial and analyzed using the HPLC.

The remaining porcine vaginal epithelial membrane was processed as follows:
(i) The epithelial membrane was placed into individual tissue homogenizer vials and 1 mL of extraction solvent was added.
(ii) The tissue homogenizer vial from Step (i) was placed in the tissue homogenizer and the contents homogenized at 5,800 RPM for 2×20 s at ambient laboratory temperature.
(iii) The contents of the tissue homogenizer vial from Step (ii) were emptied into a 7 mL glass vial.
(iv) Extraction diluent (1 mL) was added to the empty tissue homogenizer vial and the vial vortex mixed for ca. 30 s; the contents were then emptied into the glass vial from Step (iii).
(v) Each vial was then shaken on an orbital shaker at ambient temperature for 16-20 h in the extraction diluent to facilitate the extraction.
(vi) Following the extraction procedure the extraction diluent was removed from the vials and centrifuged at ca. 16,060 g-force for 10 min to remove all un-dissolved materials and particles.
(vii) The supernatant from each sample was transferred to a HPLC vial and analyzed using the HPLC.

The sums of the mean amount of the pharmacologically active compound according to the invention recovered from the epithelium of porcine vaginal tissue following application of the formulation of Example 1 after 24 h are summarized in the following table:

| Recovery of the pharmacologically active compound | | |
|---|---|---|
| Mean recovery | 9.72 | μg |
| Mean recovery | 810.21 | μg/mL |
| Total concentration presented to vaginal epithelium | 1596.04 | μM |

FIG. 1 shows the amount of pharmacologically active compound according to the invention in g recovered from the surface (not penetrated), from the porcine vaginal tissue epithelium (penetrated to the targeted tissue), and from a receiver fluid (penetrated into systemic circulation) after 24 h following application of an inventive formulation to porcine vaginal tissue. The bars represent the mean level of the pharmacologically active compound according to the invention recovered, wherein “±” means standard error of the mean, the number of repetitions was 4-6.

The experimental data illustrates that the pharmacologically active ingredient according to the invention permeates into the epithelium of porcine vaginal tissue.

1. A method for treating a subject afflicted with vulvar pain, the method comprising administering to the subject an amount of a pharmacologically active compound which is cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′ [1 H]-pyrido[3,4-b]indol]-4-amine or a physiologically acceptable salt thereof.

2. The method according to claim 1, wherein the vulvar pain is vulvodynia.

3. The method according to claim 2, wherein the vulvodynia is generalized vulvodynia, localized vulvodynia, or mixtures thereof.

4. The method according to claim 3, wherein the vulvodynia is localized vulvodynia selected from the group consisting of vestibulodynia, clitorodynia, hemivulvodynia, and pain localized in the labia minora.

5. The method according to claim 2, wherein the vulvodynia is provoked, unprovoked, or a mixture thereof.

6. The method according to claim 1, wherein the vulvar pain is related to a disorder selected from infectious disorders, inflammatory disorders, neoplastic disorders, neurologic disorders, and hormonal disbalance.

7. The method according to claim 6, wherein the pain is related to
an infectious disorder selected from the group consisting of vulvar candidiasis and herpes;
an inflammatory disorder selected from the group consisting of lichen sclerosus, lichen planus, atrophic vaginitis and immunobullous disorder;
a neoplastic disorder selected from the group consisting of Paget's disease, vulvar intraepithelial neoplasia and squamous cell carcinoma;
a neurologic disorder selected from herpes neuralgia and spinal nerve compression; or
hormonal disbalance.

8. The method according to claim 1, wherein the compound is administered topically.

9. The method according to claim 1, wherein the compound is administered locally.

10. The method according to claim 1, wherein the compound is administered on the vulva or on regions thereof.

11. The method according to claim 1, wherein after administration, the compound permeates into the epithelium of the vulvar tissue of the subject.

12. The method according to claim 1, wherein the compound is administered in form of a solution, dispersion, emulsion, suspension, or a mixture thereof.

13. The method according to claim 1, wherein the compound is formulated in form of a liquid, a gel, an ointment, a creme, or a lotion.

14. The method according to claim 1, wherein the compound is administered once, twice or three times a day.

15. The method according to claim 1, wherein cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine is present in its free base form.

16. The method according to claim 3, wherein the vulvodynia is provoked, unprovoked, or a mixture thereof.

17. The method according to claim 4, wherein the vulvodynia is provoked, unprovoked, or a mixture thereof
